(19) United States
(12) Patent Application Publication
(10) Pub. No.: US 2009/0043102 A1
(43) Pub. Date: Feb. 12, 2009

(54) METHOD FOR PRODUCING HETEROAROMATIC ALCOHOLS

(75) Inventors: Nils Bottke, Mannheim (DE); Andrea Haunert, Mannheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/063,316

(22) PCT Filed: Aug. 2, 2006
(86) PCT No.: PCT/EP06/64994

§ 371 Date: Feb. 8, 2008

(30) Foreign Application Priority Data
Aug. 11, 2005 (EP) 05107407.8

(51) Int. Cl. C07D 213/62

(52) U.S. Cl. 546/290

(57) ABSTRACT

A process for preparing heteroaromatic alcohols by catalytically hydrogenating heteroaromatic carboxylic acids or esters thereof by hydrogenating with hydrogen in the presence of a ruthenium-phosphine complex in an organic solvent.

The present invention relates to a process for preparing heteroaromatic alcohols by hydrogenating heteroaromatic carboxylic acids or esters thereof in the presence of a catalyst.

WO 03/093208 discloses a process for catalytically hydrogenating carboxylic acids in the presence of water, the catalysts described being, for example, complexes of ruthenium with tridentate phosphines.

JP 2004-300131 A discloses a process for preparing alcohols by hydrogenating carboxylic esters in the presence of ruthenium complex catalysts with phosphine ligands.

H. T. Teunissen et al. describe the hydrogenation of dimethyl phthalate over a homogeneous ruthenium catalyst with phosphine ligands (Chem. Commun. 1998, p. 1367-1368).

The hydrogenation of heteroaromatic carboxylic acids and esters thereof to the corresponding alcohols is effected, according to the prior art, in particular by reduction with complex hydrides such as LiAlH4 or NaBH4 (K. Soai et al., Synth. Commun. 1982, 12, 463-468).

However, a problem in the hydrogenation of heteroaromatic compounds is that they tend to ring hydrogenation compared to the carbocyclic analogs.

It was an object of the present invention to find an improved process for preparing heteroaromatic alcohols from heteroaromatic carboxylic acids or esters thereof.

Accordingly, a process has been found for preparing heteroraromatic alcohols by catalytically hydrogenating heteroaromatic carboxylic acids or esters thereof, which comprises hydrogenating with hydrogen in the presence of a rutheniuin-phosphine complex in an organic solvent.

Useful heteroaromatic carboxylic acids and esters thereof are, in accordance with the invention, compounds of the general formula A—CO₂R where A is a monocyclic five- to seven-membered heteroaromatic radical which has from one to two heteroatoms and may optionally be substituted, and R is H or linear and branched C₁—C₁₂-alkyl. Useful heteroatoms include nitrogen and sulfur, preferably nitrogen. Suitable substituents on the heteroaryl radical are: H, alkyl, aryl, —OR, —NR₂, halogen such as —F, —Cl, —Br, preferably methyl or methoxy.

Preferred reactants are pyridine-2-carboxylic acid, pyridine-3-carboxylic acid, pyridine-4-carboxylic acid and the corresponding linear or branched C₁—C₁₂-alkyl esters, preferably methyl, ethyl, propyl or isopropyl esters.

A useful complex ligand is preferably a phosphine ligand of the general formula R¹—CQ₃ in which Q may be the same or different and is a —R²PR³R⁴ group, where R¹ is H, C₁—C₄-alkyl or aryl, R² is (CH₂)ₙ where n=1-4, and where R³, R⁴ may be the same or different and each an aryl, cyclohexyl or tert-butyl radical.

R¹ may, for example, be methyl or ethyl, preference being given to methyl. R² is preferably a methylene diradical. R³, R⁴ are preferably each phenyl. A particularly preferred ligand is tris(diphenyiphosphinomethyl)ethane.

The preparation of such ruthenium-phosphine complexes is known per se. It can be effected by reacting a trivalent ruthenium compound with the selected phosphine ligands. The complex is preferably prepared in situ. Typically from 1.0 to 1.5 mol of phosphine are used per mole of ruthenium compound.

The ruthenium compounds used may, for example, be RuCl₃ or ruthenium triacetylacetonate (Ru(acac)₃). Preference is given to using ruthenium triacetylacetonate.

In the ruthenium-phosphine complexes, one molecule of the phosphine and two molecules of acetylacetonate are present bonded to one ruthenium atom.

The hydrogenation can be effected over a homogeneous catalyst or an immobilized catalyst. The catalyst may, for example, be bound to a polymer. Preference is given to homogeneous catalysis.

Typically, from 0.001 to 0.2 mol, preferably from 0.002 to 0.017 mol, of catalyst is used per mole of heteroaromatic carboxylic acid or ester.

The hydrogenation is effected in the presence of elemental hydrogen at a hydrogen pressure of from 1 to 30 MPa, preferably from 5 to 20 MPa, more preferably from 12 to 17 MPa.

The reaction temperature may be from 25 to 250° C., preferably from 100 to 200° C., more preferably from 100 to 150° C.

The reaction times are guided by the amount of the compounds used and are typically in the range from 12 to 96 hours, preferably from 24 to 48 hours.

Suitable solvents are organic solvents, preferably alcohols, more preferably secondary alcohols, most preferably isopropanol, trifluoroisopropanol and hexafluoroisopropanol.

The preparation can be effected continuously or batchwise, preferably batchwise.

The preparation can be effected in any reactor suitable for a high-pressure hydrogenation. Typically, the reactant is initially charged in the selected solvent and then either the separately prepared catalyst is added or, in the case of catalyst preparation in situ, the ruthenium(III) compound and the phosphine ligand. Subsequently, the reaction mixture is heated to the desired reaction temperature and the desired hydrogen pressure is established. After the reaction has ended, the reactor is decompressed and the process product is worked up in a manner customary per se, for example by removing the solvent by distillation and subsequently working up the crude product by vacuum distillation.

The process according to the invention can afford the corresponding hydroxymethylheteroaryl compounds of the formula A—CH₂OH in good yields and with good selectivity.

### EXAMPLES

In an autoclave, the reactant and Ru(acac)₃ were dissolved with 1.4 equivalents of tris(diphenylphosphinomethyl)ethane in isopropanol, and converted at 150° C. and 150 bar of hydrogen pressure for 24 h. After the reaction, the reaction mixture was analyzed by gas chromatography.

Conversion, selectivity and yield were determined by means of gas chromatography. Substrates, batch sizes and analysis are compiled in Table 1.

| | | | | Con- | | | |
|---|---|---|---|---|---|---|---|
| | | | Cat. | version | Yield | Sel. | |
| | Substrate | mmol | [mol %] | [%] | [%] | [%] | ^{a)} |
| 1 | Ethyl isonicotinate | 45 | 1.0 | 100 | 97.7 | 97.7 | A |
| 2 | Ethyl isonicotinate | 45 | 0.4 | 90.8 | 89.5 | 98.6 | A |
| 3 | Ethyl isonicotinate | 24 | 0.2 | 77.9 | 74.5 | 95.6 | B |
| 4 | Isopropyl isonicotinate | 45 | 0.4 | 74.1 | 72.3 | 97.6 | A |
| 5 | Methyl nicotinate | 45 | 1.0 | 92.3 | 63.5 | 68.8^{b)} | A |
| 6 | Methyl nicotinate | 3 | 1.7 | 91.9 | 60.5 | 65.8^{b)} | B |

^{a)}A = 150 ml of isopropanol, 300 ml autoclave; B = 20 ml of isopropanol, 50 ml autoclave.
^{b)}Selectivity loss via ring hydrogenation as a side reaction.

1-9. (canceled)

10. A process for preparing heteroraromatic alcohols comprising catalytically hydrogenating heteroaromatic carboxylic acids or esters thereof with hydrogen in the presence of a rutienium-phosphine complex in an organic solvent.

11. The process of claim 10, wherein said heteroaromatic carboxylic acids or esters thereof are compounds of general formula A—CO₂R, wherein A is a monocyclic five-to seven-membered aromatic radical having from one to two heteroatoms and R is a linear or branched C₁—C₁₂-alkyl radical.

12. The process of claim 10, wherein said heteroaromatic carboxylic acids or esters thereof are pyridinecarboxylic acids or esters thereof

13. The process of claim 10, wherein said ruthenium-phosphine complex is prepared by reacting a ruthenium(III) compound with a phosphine ligand.

14. The process of claim 10, wherein said ruthenium-phosphine complex is homogeneous.

15. The process of claim 10, wherein said ruthenium-phosphine complex comprises a phosphine ligand of general formula R¹—CQ₃; wherein R¹ is H, C₁—C₄-alkyl, or aryl; wherein Q is the same or different and is a —R²PR³R⁴ group; wherein R² is (CH₂)ₙ, wherein n is 1, 2, 3, or 4; and wherein R³ and R⁴ are, identically or differently, an aryl, cyclohexyl, or tert-butyl radical.

16. The process of claim 15, wherein said phosphine ligand is tris(diphenylphosphinomethyl)ethane.

17. The process of claim 10, wherein said organic solvent is a secondary alcohol.

18. The process of claim 17, wherein said secondary alcohol is isopropanol, trifluoroisopropanol, or hexafluoroisopropanol.
